(19) United States
(12) Plant Patent Application Publication
(10) Pub. No.: US 2003/0074703 P1
(43) Pub. Date: Apr. 17, 2003

(54) Strawberry plant named El Capitan

(76) Inventors: Amado Q. Amorao, Camarillo, CA (US); Arnoldo Solis JR., Oxnard, CA (US); Michael Ferguson, Chatsworth, CA (US)

Correspondence Address: PENNIE & EDMONDS LLP, COUNSELLORS AT LAW, 1155 Avenue of the Americas, New York, NY, 10036-2711, US

(21) Appl. No.: 09/968,563

(22) Filed: Oct. 1, 2001

(51) Int. Cl. A01H 5/00

(52) U.S. Cl. PLT/209

(57) ABSTRACT

This invention relates to a new and distinct variety of strawberry named El Capitan. The variety is similar to the varietics Coronado and San Miguel. The variety is distinguished from Coronado and San Miguel, in that El Capitan has an obtuse terminal leaflet teeth shape, medium to dense petiole pubescence, medium to strong anthocyanin coloration, cordate fruit shape, marked difference in shape between primary and secondary fruits, large hollow center size, and time of flowering is mid-December.

### BACKGROUND OF THE INVENTION

The new variety originated as a result of a controlled cross between the strawberry plants San Miguel (U.S. Plant Pat. No. 10,642) and 12A71 (unpatented variety) in an ongoing breeding program, and was discovered as a seedling in Ventura County, Calif. in February, 1996. The original seedling of the new cultivar was asexually propagated by stolons in a nursery in Shasta County, Calif. Propagules were transplanted to a controlled breeding plot in Ventura County, Calif., where the variety was identified and selected for further evaluation. El Capitan was subsequently asexually propagated and underwent firther testing at in Ventura County, Calif. for six years. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct variety of strawberry named El Capitan. The variety is botanically identified as Fragaria×ananassa. The new variety is distinguished from other varieties by a number of characteristics as set forth in Tables 1-4.

### COMPARISON TO SIMILAR VARIETIES

The varieties which we believe to be similar to El Capitan from those known to us are Coronado (U.S. Plant Pat. No. 10,221) and San Miguel (U.S. Plant Pat. No. 10,642). There are several characteristics of the new variety that are different from, or not possessed by Coronado and San Miguel. The new variety has an obtuse terminal leaflet teeth shape, medium to dense petiole pubescence, medium to strong anthocyanin coloration, cordate fruit shape, marked difference in shape between primary and secondary fruits, large hollow center size, and time of flowering is mid-December.

### BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows the leaves of the plant.

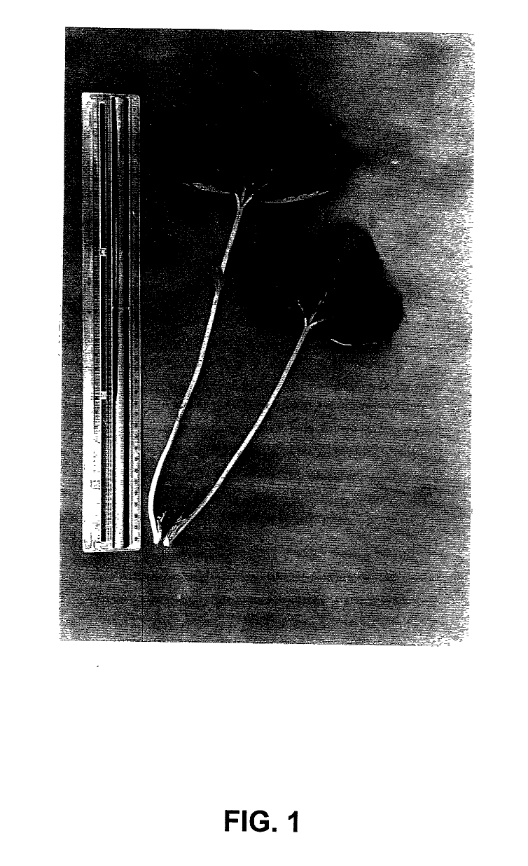

FIG. 2 shows the upper side and the under side of the flowers.

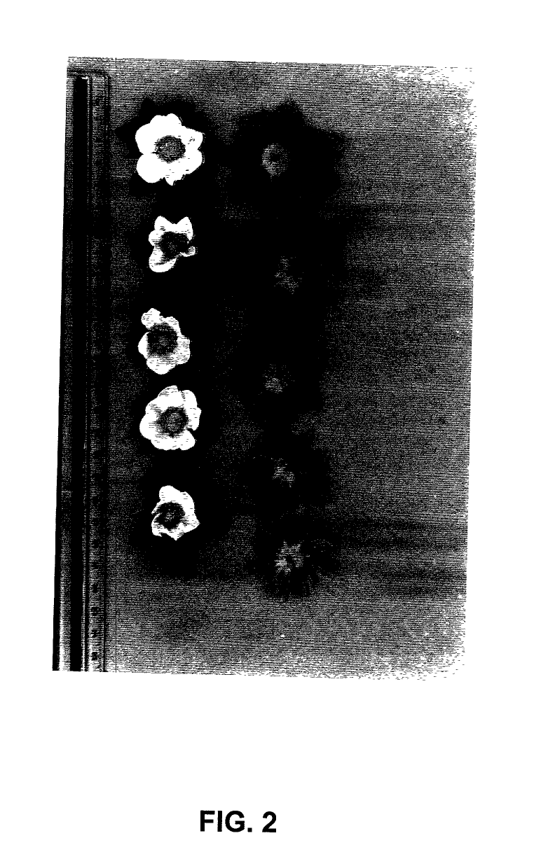

FIG. 3 shows a close-up of the fruit.

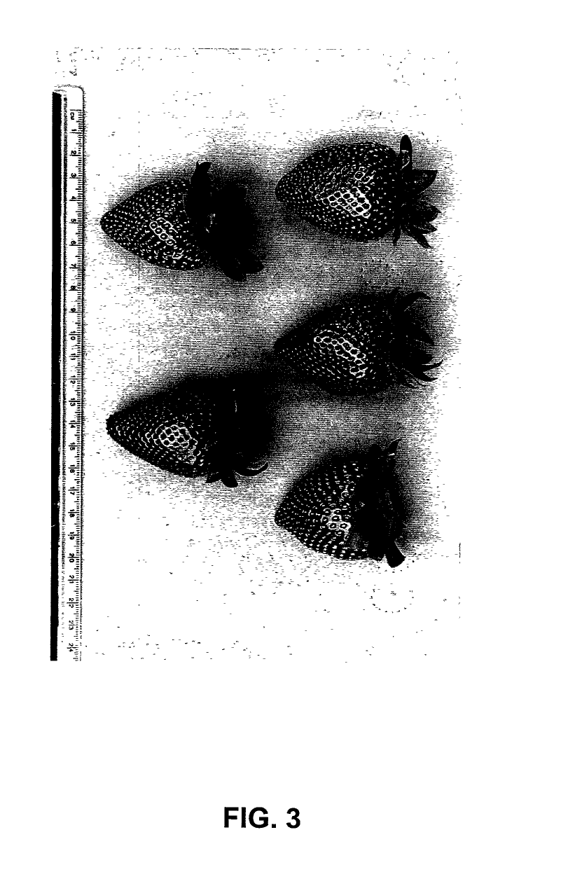

FIG. 4 shows the fruit in longitudinal cross-section.

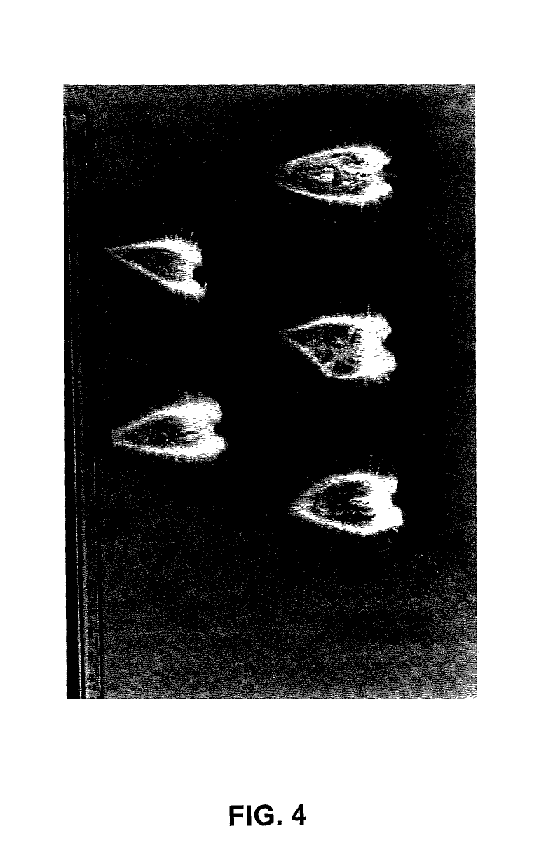

### DESCRIPTION OF THE NEW VARIETY

The following detailed description of the new variety is based upon observations taken of plants and fruit grown in Monterey County, Calif., U.S.A. This description is in accordance with UPOV terminology. Observations of El Capitan, Coronado and San Miguel were taken in side by side comparison in the year 2001. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### PROPAGATION

The new variety is principally propagated by way of stolons. Although propagation by stolons is presently preferred, other known methods of propagating strawberry plants may be employed.

### CHARACTERISTICS OF THE NEW VARIETY

Information on the new variety is presented in Tables 1, 2, 3 and 4. In the tables, the flowers described are secondary flowers except where indicated. The petal color of El Capitan is white (155C in the RHS Colour Chart). The fruit described is the secondary fruit on one-year-old plants. Fruit and flower measurements are an average of both primary and secondary fruit and flowers.

Table 1 provides information on the plant and fruit characteristics of the new variety El Capitan compared with characteristics of Coronado and San Miguel. Table 2 provides additional information of the plant and fruit characteristics of the new variety El Capitan compared with characteristics of the varieties Coronado and San Miguel. Table 3 provides reactions of the new variety to stresses, pests and diseases compared with reactions of the varieties Coronado and San Miguel. Table 4 provides isozyme characteristics of the new variety as compared to that of the varieties Coronado and San Miguel.

TABLE 1
DETAILED COMPARISON OF EL CAPITAN, CORONADO AND
SAN MIGUEL

| | El Capitan | Coronado | San Miguel |
|---|---|---|---|
| Plant Characteristics | | | |
| Height of Plant (cm) | 24.0 | 20.7 | 22.8 |
| Spread of Plant (cm) | 32.6 | 28.7 | 29.4 |
| Number of Crowns | 6.0 | 4.6 | 4.4 |
| Leaf Characteristics | | | |
| Terminal Leaflet | 6.0 | 6.1 | 6.2 |
| Length (cm) | | | |
| Terminal Leaflet | 6.0 | 6.3 | 5.8 |
| Width (cm) | | | |
| Terminal Leaflet | 1.00 | 0.97 | 1.07 |
| Length/Width | | | |
| # Teeth/Terminal | 16.3 | 21.6 | 20.7 |
| Leaflet | | | |
| Color of upper side | dark green | light to | medium to |
| | 147A | medium green | dark green |
| | | 137A | 147A |
| Color of under side | light to | light to | light to |
| | medium green | medium green | medium green |
| | 147C | 137B | 138B |
| Petiole Length (cm) | 18.2 | 16.3 | 16.0 |
| Petiole Color | 149A | 149A | 149A |
| Bract Frequency | 8% | 0% | 30% |
| | typically | | typically |
| | single | | paired |
| Stipule Length (cm) | 3.4 | 3.4 | 2.9 |
| Stipule Width (cm) | 1.1 | 1.2 | 1.1 |
| Flower Characteristics | | | |
| Petal Length (cm) | 1.5 | 1.6 | 1.4 |
| Petal Width (cm) | 1.7 | 1.7 | 1.5 |
| Petal Length/Width | 0.91 | 0.93 | 0.98 |
| Ratio | | | |
| Flower Diameter (cm) | 3.2 | 3.4 | 2.7 |
| Calyx Diameter (cm) | 4.4 | 4.4 | 4.1 |
| Fruiting Truss | | | |
| Length (cm) | 22.9 | 22.1 | 25.7 |
| Fruit Characteristics | | | |
| Fruit Length (cm) | 5.0 | 4.3 | 4.3 |
| Fruit Width (cm) | 3.9 | 3.9 | 4.1 |
| Fruit Length/Width | 1.28 | 1.11 | 1.07 |
| Ratio | | | |
| Average Berry Weight | 23.3 | 23.6 | 23.2 |
| (g) | | | |
| External Color | dark red | red | dark red |
| | 53A | 46A | 53A |
| Internal Color | red | red | dark red |
| | 43A | 41A | 45A |
| Average % brix | 7.9 | 7.1 | 7.1 |
| Brix/Acid Ratio | 9.71 | 9.11 | 9.33 |
| Achene Coloration | dark red to | dark red to | dark red to |
| | yellow green | yellow | yellow green |
| | 60A to 151A | 53A to 141B | 53A to 151B |
| Yield (g/plant) | 695 | 598 | 557 |

TABLE 2
CHARACTERISTICS OF EL CAPITAN, CORONADO AND
SAN MIGUEL

| | El Capitan | Coronado | San Miguel |
|---|---|---|---|
| Plant | | | |
| Habit | globose | globose | flat globose |
| Density | open | open | open |
| Vigor | strong | medium | medium |
| Leaf | | | |
| Shape in cross section | concave | slightly | concave |
| | | concave | |
| Interveinal blistering | medium | strong | strong |
| Glossiness | medium strong | strong | strong |
| Number of leaflets | three only | more than three | three only |
| | | on up to 5 out | |
| | | of 10 leaves | |
| Terminal leaflet | flat | flat to cupped | flat to revolute |
| margin | | | |
| Terminal leaflet | obtuse | obtuse | rounded |
| shape of base | | | |
| Terminal leaflet | obtuse | rounded | rounded |
| shape of teeth | | | |
| Stipule pubescence | sparse | sparse | sparse |
| Petiole pubescence | medium to | medium | sparse to |
| | dense | | medium |
| Petiole pose of hairs | outward | outward | outward |
| Stolon | | | |
| Amount | many | medium to | many |
| | | many | |
| Anthocyanin | medium to | weak to | weak to |
| coloration | strong | medium | medium |
| Thickness | thin | medium | thin to medium |
| Pubescence | sparse to | sparse to | sparse to |
| | medium | medium | medium |
| Inflorescence | | | |
| Position relative to | above | level to above | above |
| foliage | | | |
| Diameter of calyx | larger | larger | larger |
| relative to corolla on | | | |
| secondary flowers | | | |
| Diameter of inner | smaller to same | same size to | same size |
| calyx relative to outer | size | larger | |
| on secondary flowers | | | |
| Spacing of petals | overlapping | touching to | overlapping |
| | | overlapping | |
| Fruiting Truss | | | |
| Attitude at first | prostrate | semi-erect to | prostrate |
| picking | | prostrate | |
| Fruit | | | |
| Predominant shape | cordate | conical to | conical to |
| | | wedged | cylindrical |
| Difference in shapes | marked | moderate | moderate |
| between primary and | | | |
| secondary fruits | | | |
| Band without achenes | very narrow | narrow | very narrow |
| Unevenness of surface | weak to | weak to | weak to |
| | medium | medium | medium |
| Evenness of color | slightly uneven | even | even |
| uneven | to even | | |
| Glossiness | strong | medium to | medium |
| | | strong | |
| Insertion of achenes | below to level | below surface | below surface |
| | with the | | |
| | surface | | |
| Insertion of calyx | in a basin to | level to set | set above |
| | level | above | |
| Pose of the calyx | reflexed | spreading | spreading to |
| segments | | | reflexed |
| Size of calyx in | larger | larger | same size |
| relation to fruit on | | | |
| secondary fruit | | | |
| Adherence of calyx | strong | medium | strong |
| Firmness of flesh | firm | firm | firm |
| Evenness of flesh | even | slightly uneven | even |
| color | | | |
| Distribution of flesh | marginal and | marginal and | marginal and |
| color | central | central | central |
| Hollow center size | large | medium | medium |
| Sweetness | strong | weak | strong |
| Texture when tasted | fine | fine | fine |
| Acidity | medium | strong | medium |
| Time of Flowering | mid-December | early-January | early-January |
| Harvest Interval in | late-January to | early-February | early-February |
| 2001 | mid-June | to mid-June | to mid-June |
| Type of Bearing | partially | partially | partially |
| | everbearing | everbearing | everbearing |

TABLE 3

| | El Capitan | Coronado | San Miguel |
|---|---|---|---|
| Reaction to Stress | | | |
| high pH | moderately | moderately | moderately |
| | resistant | resistant | resistant |
| high soil salt levels | moderately | moderately | moderately |
| | resistant | resistant | resistant |
| Reaction to Pests | | | |
| Tetranychus urticae | moderately | moderately | moderately |
| | resistant | resistant | resistant |
| Lygus hesperus | susceptible | susceptible | susceptible |
| Reaction To Diseases | | | |
| Botrytis fruit rot | moderately | moderately | moderately |
| | susceptible | susceptible | susceptible |
| Powdery mildew | moderately | moderately | moderately |
| | resistant | resistant | resistant |
| Verticillium wilt | susceptible | susceptible | susceptible |
| Strawberry Mottle Virus | moderately | moderately | moderately |
| | resistant | resistant | resistant |
| Xanthomonas fragariae | moderately | moderately | moderately |
| | susceptible | susceptible | susceptible |

### ISOZYME ANALYSIS

In addition to the morphological description above, the new cultivar El Capitan has been analyzed to obtain an indication of its genetic makeup to provide further means for identifying the new variety and distinguishing it from other somewhat similar and/or related strawberry varieties. Specifically, leaf samples of El Capitan, Coronado, and San Miguel were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR EL CAPITAN, CORONADO AND
SAN MIGUEL

| Locus | El Capitan | Coronado | San Miguel |
|---|---|---|---|
| PGI | A2 | A2 | A4 |
| LAP | B3 | B3 | B3 |
| PGM | C4 | C4 | C2 |

What is claimed is:

1. A new and distinct variety of strawberry plant, substantially as shown and described.
